(19) United States
(12) Patent Application Publication
(10) Pub. No.: US 2003/0135142 A1
(43) Pub. Date: Jul. 17, 2003

(54) Safety device for prevention of carpal tunnel syndrome

(76) Inventor: Richard C. Reinhardt, Granite City, IL (US)

Correspondence Address: OSTROLENK FABER GERB & SOFFEN, 1180 AVENUE OF THE AMERICAS, NEW YORK, NY, 100368403

(21) Appl. No.: 10/345,431

(22) Filed: Jan. 14, 2003

Related U.S. Application Data
(60) Provisional application No. 60/348,915, filed on Jan. 15, 2002.

(51) Int. Cl. A61F 5/00

(52) U.S. Cl. 602/21

(57) ABSTRACT

A device to prevent and treat carpal tunnel syndrome that restricts wrist extension to a maximum of about 20 degrees, while allowing a user his or her full range of wrist flexion motion. The device may comprise a wrist portion that receives the wrist, a dorsal portion that includes a rigid or possibly flexible insert, and two eyelets through which the second and third fingers are received.

### RELATED APPLICATION

The application is based on and claims priority to U.S. Provisional Application No. 60/348,915, filed Jan. 15, 2002, entitled SAFETY DEVICE FOR PREVENTION OF CARPAL TUNNEL SYNDROME, of which the entire disclosure is incorporated herein by reference.

### FIELD OF THE INVENTION

The present invention is related to ergonomics, and more particularly, is directed to an ergonomic flexible brace for people with carpal tunnel syndrome.

### BACKGROUND OF THE INVENTION

Thousands of people suffer from carpal tunnel syndrome. Carpal tunnel syndrome is an ailment that affects the median nerve of a person's wrist due to pressure through an opening in the wrist called a carpal tunnel. The condition causes many uncomfortable symptoms such as pain, tingling and numbness in the hands and arms. A combination of poor wrist posture and repetitive motion is a classic cause for carpal tunnel syndrome. For many sufferers of carpal tunnel syndrome, relief comes in the form of overuse of medications, for example, prescription and over the counter pain relievers.

Many carpal tunnel syndrome sufferers decide to abandon their chosen professions in order to find relief from the chronic discomfort brought on by the syndrome. Some of the most acute forms of carpal tunnel syndrome cause people to suffer total disruption of their personal and professional lives. Moreover, chronic sufferers may have to endure repeated surgeries that may provide relief only for the short term or, in some cases, not at all.

A large number of commercial devices, for example, braces and splints, are available that purport to treat and prevent carpal tunnel syndrome. Such devices attempt to fix the wrist in a single position, or, alternatively, restrict the wrist to a range of motion that manufacturers consider not to cause carpal tunnel syndrome. Unfortunately, restricting the wrist to a fixed position or limiting the wrist to such a narrow range of motion is frequently ineffective in the treatment and prevention of carpal tunnel syndrome. For example, the range of wrist motion may be too large, and/or the limited range of wrist motion may not help to cure or prevent carpal tunnel syndrome.

Many of the devices used to avoid or relieve pain contribute to secondary complications. For example, if motion of the wrist is restricted, motion-related injuries may occur at the elbow, shoulder, neck and fingers. Such devices are ergonomically inefficient. Injuries and accidents in the work place are often directly attributed to devices that restrict motion. Additionally, splinting devices that strictly reduce range of motion may cause further disabilities because such devices precipitate “disuse atrophy.” Moreover, many prior art motion restricting devices cause a number of complications in performing tasks associated with the work place and in the performance of critical movements not necessarily associated with work.

### SUMMARY OF THE INVENTION

The present invention is directed to providing treatment for carpal tunnel syndrome without causing the many problems associated above with prior art devices. The present invention deals efficiently with the pain associated with carpal tunnel syndrome and simultaneously reduces the potential risks and problems described above.

Many instances of carpal tunnel syndrome occur because of excessive extension of the wrist. More particularly, wrist extension over 20° is considered harmful and likely to cause carpal tunnel syndrome. Conversely, wrist flexion is not considered harmful and does not cause damage to the carpal tunnel, such as carpal tunnel syndrome. Poor wrist posture (i.e., wrist extension above 20°) leads to inflammation of the carpal tunnel and the eventual development of carpal tunnel syndrome.

The present invention addresses the cause of carpal tunnel syndrome and reduces pain and other problems associated with carpal tunnel syndrome by controlling wrist posture. More specifically, the invention relates to a device that comprises a wrist portion which forms a channel through which a person's wrist is received, the wrist portion having a first end and a second end, a dorsal portion having a first end that extends from the second end of the wrist portion, a rigid insert received in a sleeve in the dorsal portion, and a finger receiving portion of the dorsal portion at a second end of the dorsal portion.

The device does not impede flexion motion and, therefore, helps to maintain essential blood circulation to the wrist, hand and fingers and, therefore, reduces the possibility of secondary complications, such as compression injuries and blood circulatory problems. Wrist extension over about 20°, however, is restricted by the present invention. Therefore, the likelihood of developing carpal tunnel syndrome due to extension of the wrist over 20° is addressed, while the likelihood of secondary complications and/or associated clumsiness from restrictions of movement, such as flexion, is severely reduced.

### DETAILED DESCRIPTION OF THE DRAWINGS

For the purposes of illustrating the invention, there are shown in the drawings embodiments which are presently preferred, it being understood, however, that the invention is not limited to the precise arrangements and instrumentalities shown. The features and advantages of the present invention will become apparent from the following description of the invention which refers to the accompanying drawings, in which:

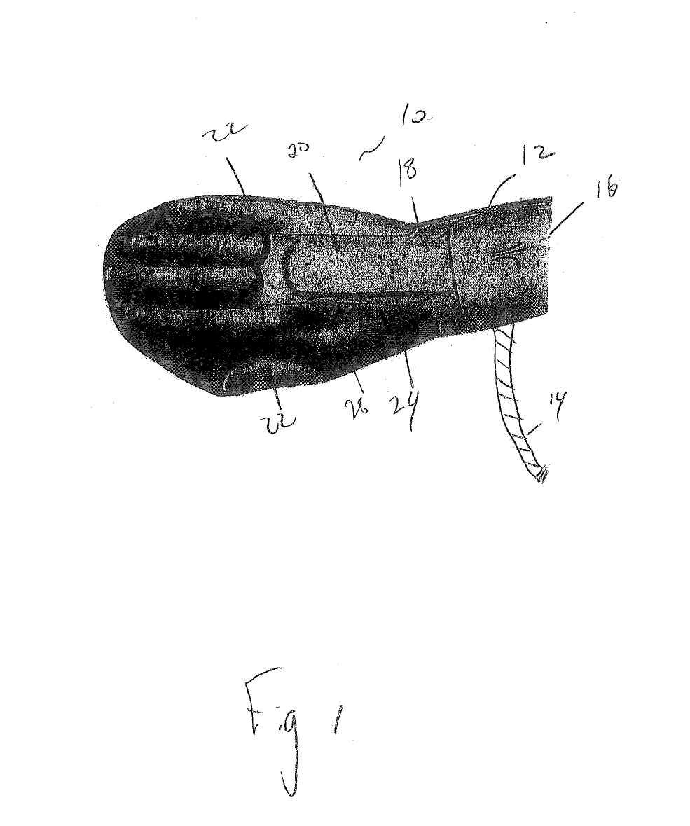

FIG. 1 is a top view of a first embodiment of the present invention having a rigid insert and shown being worn on a person's wrist;

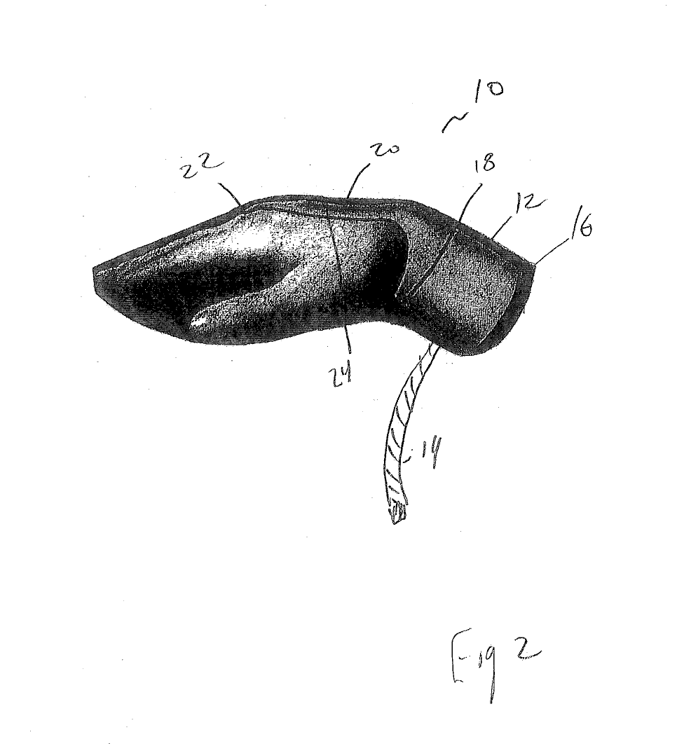

FIG. 2 is a side view of a second embodiment of the invention having a flexible insert and illustrates the wearer's ability to flex the wrist; and

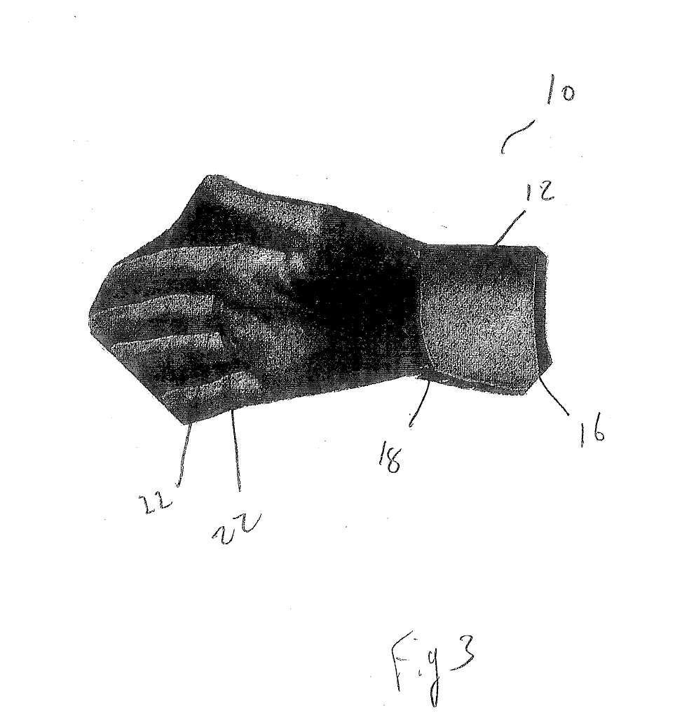

FIG. 3 is a bottom view of a third embodiment of the present invention and shown being worn on a person's wrist.

### DETAILED DESCRIPTION OF EMBODIMENTS OF THE INVENTION

Referring now to the figures, in which like reference numerals refer to like elements, there is shown in FIG. 1 an example of a device in accordance with the present invention and referred to herein, generally, as carpal tunnel syndrome prevention device 10.

In a preferred embodiment, the device 10 comprises a wrist portion 12 that is made from a flexible body conforming material, for example, LYCRA, and through which a person's wrist is received. In the example shown in FIG. 1, the wrist portion 12 also includes a strap 14 that wraps around the lower or upper side of the wrist and fastens via a fastening material, such as VELCRO, a button, hook, snap or the like. The strap 14 enables the wearer to adjust the tension of the wrist portion 12 while worn on the wrist.

As shown in FIG. 1, the wrist portion 12 further comprises a first end 16 and a second end 18. The second end 18 meets an elongated dorsal portion 20 that extends over the dorsal part of the wearer's hand. In the embodiment shown in FIG. 1, the dorsal portion 20 terminates at two eyelets 22, through which the second and third fingers of the wearer's hand are preferably inserted.

Furthermore, the dorsal portion 20 preferably houses an insert 24. Preferably, the insert 24 has dimensions that are approximately one inch wide by four inches long, and is made from a very inflexible material, for example, a hard plastic, metal, rubber, fiberglass or other rigid material. A combination of such inflexible materials can also be used to construct the insert 24. The insert 24 assists in the device 10 to prevent undesirable motion of the wrist, for example, wrist extension over 20°.

In a preferred embodiment of the present invention, the dorsal portion 20 comprises a sleeve 26 that receives the insert 24. The sleeve 26 preferably is open at one end that enables the insert 24 to be inserted and removed at will. The sleeve 26 can further be outfitted with VELCRO, a button, snap or the like to close the open end and enclose the insert 24 securely in the sleeve. In an alternative embodiment, the sleeve 26 is fully enclosed and the insert 24 is, for example, sewn into the carpal tunnel prevention device 10.

Since the insert 24 is housed in the dorsal portion 20 of the carpal tunnel prevention device 10, only wrist extension is prevented, while wrist flexion is mostly unaffected.

FIG. 2 shows a side view of a second embodiment of the present invention. Some features of the second embodiment are common to the first embodiment shown in FIG. 1, as follows. As shown in FIG. 2, flexion of the wrist is not significantly impeded by the device 10. Also as shown in FIG. 2, most of the hand is not covered by the device so the skin of the hand can “breathe”.

As noted above, a significant cause of carpal tunnel syndrome is extension of the wrist over 20°. In the second embodiment, unlike the first embodiment, the insert 24 that is placed in the carpal tunnel prevention device 10 is somewhat flexible. FIG. 2 shows flexing of the insert 24 in response to the flexion movement of the user's wrist. The insert may be made of any suitable flexible material, for example plastic or rubber; and further may be a somewhat inflexible material that is molded with a “living hinge.” However, in any case, the insert 24 is sufficiently stiff to be effective at preventing the wearer from extending his or her wrist beyond the dangerous 20° extension position. However, the wearer's wrist is not restricted from flexion movement, in contrast with prior art devices.

FIG. 3 shows an alternative embodiment of the present invention. FIG. 3 shows a bottom view of the device 10 and, further, illustrates the eyelets 22 through which the second and third fingers of the wearer's hand are preferably inserted. The strap 14 is not included in the embodiment shown in FIG. 3, and the wrist portion 12 is a single, enclosed piece that receives the wrist. Preferably, the wrist portion 12 is a fully enclosed, single piece that is capable of receiving a person's wrist. In such case, the wrist portion 12 is elastic and adjusts to dimensions of the wearer's wrist. Moreover as shown in FIG. 3, the palm and the fingers of the wearer's hand are fully exposed. The wearer's hand's gripping movements are not hindered by the device 10, and the likelihood of nerve restriction and compression-type damage is significantly reduced thereby. The placement of the insert 12 in the dorsal portion 18 of the device 10 restricts extension motion, especially beyond 20°.

Thus, according to the foregoing embodiments, the device 10 is effective to prevent and treat carpal tunnel syndrome. Wrist extension over about 20°, a frequent cause of carpal tunnel syndrome, is effectively restricted, while flexion of the wrist is not impeded. Further, wearing the device reminds the user to improve the condition of his or her working conditions, such as the position of a computer keyboard, a computer pointing device and the like. Moreover, by preventing compression type injuries, the present invention serves to maintain blood circulation to the wrist, hand and fingers and, therefore, reduces the possibility of secondary complications. Wrist flexion is unimpeded, while wrist extension over 20° is restricted by the present invention. Therefore, the likelihood of developing carpal tunnel syndrome is prevented by the present invention and those suffering with the syndrome will benefit by wearing the device 10.

Although the present invention has been described in relation to particular embodiments thereof, many other variations and modifications and other uses will become apparent to those skilled in the art. Therefore, the present invention is not to be limited by the specific disclosure herein.

What is claimed is:

1. A device for preventing carpal tunnel syndrome, said device comprising:
a wrist portion forming a channel through which a user's wrist is received, said wrist portion having a first end and a second end;
a dorsal portion comprising a sleeve, said dorsal portion having a first end that extends from said second end of said wrist portion;
an insert disposed in said sleeve; and
a finger receiving portion at a second end of said dorsal portion and being adapted to receive at least one finger of the user,
wherein said insert is arranged in said device so as to restrict wrist extension motion more than wrist flexion motion.

2. The device of claim 1, further comprising at least one strap coupled to said wrist portion.

3. The device of claim 1, wherein said insert is rigid.

4. The device of claim 3, wherein said insert is made of at least one of plastic, metal, rubber and fiberglass.

5. The device of claim 1, wherein said insert is flexible.

6. The device of claim 5, wherein said insert is made of at least one of plastic and rubber.

7. The device of claim 1, wherein said sleeve comprises a at least one open end that is adapted to receive said insert.

8. The device of claim 1, wherein said insert is enclosed in said sleeve.

9. The device of claim 1, wherein said rigid insert is approximately four inches long and approximately one inch wide.

10. The device of claim 1, wherein extension of the wearer's wrist is restricted to a maximum of about 20 degrees.

11. The device of claim 1, wherein said wrist portion is made of a body conforming material.

12. The device of claim 11, wherein the body conforming material is LYCRA.

13. A method for preventing carpal tunnel syndrome, said method comprising restricting wrist extension to a maximum of about 20 degrees.

14. A method for preventing of carpal tunnel syndrome, said method comprising the step of providing a device on the user's hand and wrist, the device having:
a) a wrist portion forming a channel through which the user's wrist is received, said wrist portion having a first end and a second end;
b) a dorsal portion comprising a sleeve, said dorsal portion having a first end that extends from said second end of said wrist portion;
c) an insert disposed in said sleeve; and
d) a finger receiving portion at a second and of said dorsal portion and being adapted to receive at least one finger of the user,
wherein said insert is arranged in said device so as to restrict wrist extension motion more than wrist flexion motion.
